(19) United States
(12) Patent Application Publication
(10) Pub. No.: US 2025/0025320 A1
(43) Pub. Date: Jan. 23, 2025

(54) STENT WITH DYNAMIC ANTI-MIGRATION PROPERTIES

(71) Applicant: BOSTON SCIENTIFIC SCIMED, INC., Maple Grove, MN (US)

(72) Inventor: Martyn G. Folan, Loughrea (IE)

(73) Assignee: BOSTON SCIENTIFIC SCIMED, INC., Maple Grove, MN (US)

(21) Appl. No.: 18/774,438

(22) Filed: Jul. 16, 2024

Related U.S. Application Data
(60) Provisional application No. 63/514,015, filed on Jul. 17, 2023.

(51) Int. Cl. A61F 2/852; A61F 2/90

(52) U.S. Cl. CPC A61F 2/852; A61F 2/90; A61F 2210/0076; A61F 2250/0036; A61F 2250/0039

(57) ABSTRACT

A medical stent includes a central stent segment, a proximal stent segment adapted to be positioned over a proximal region of the central stent segment, and a distal stent segment adapted to be positioned over a distal region of the central stent segment. A polymeric coating extends over the proximal stent segment, the distal stent segment and at least a portion of the central stent segment, the polymeric coating adapted to allow the proximal stent segment to float over the proximal region of the central stent segment and to allow the distal stent segment to float over the distal region of the central stent segment. Upon implantation, the central stent segment is adapted to translate relative to the proximal stent segment and the distal stent segment in response to peristaltic forces.

### CROSS REFERENCE TO RELATED APPLICATIONS

The present application claims the benefit of and priority to U.S. Provisional Patent Application Ser. No. 63/514,015, filed on Jul. 17, 2023, the disclosure of which is incorporated herein by reference.

### TECHNICAL FIELD

The present disclosure relates generally to methods and apparatuses for various digestive ailments. More particularly, the disclosure relates to different configurations and methods of manufacture and use of a stent.

### BACKGROUND

Implantable stents are devices that are placed in a body structure, such as a blood vessel, esophagus, trachea, biliary tract, colon, intestine, stomach or body cavity, to provide support and to maintain the structure open. These devices are manufactured by any one of a variety of different manufacturing methods and may be used according to any one of a variety of methods. Of the known medical devices, delivery systems, and methods, each has certain advantages and disadvantages. There is an ongoing need to provide alternative medical devices and delivery devices as well as alternative methods for manufacturing and using medical devices and delivery devices.

### SUMMARY

The disclosure is directed to several alternative designs, materials and methods of manufacturing medical device structures and assemblies, and the use thereof. An example may be found in a medical stent. The medical stent includes a central stent segment including a proximal region and a distal region, a proximal stent segment that is adapted to be positioned over the proximal region of the central stent segment, and a distal stent segment that is adapted to be positioned over the distal region of the central stent segment. A polymeric coating extends over the proximal stent segment, the distal stent segment and at least a portion of the central stent segment, the polymeric coating adapted to allow the proximal stent segment to float over the proximal region of the central stent segment and to allow the distal stent segment to float over the distal region of the central stent segment. Upon implantation, the proximal stent segment and/or the distal stent segment are adapted to translate relative to the central stent segment in response to peristaltic forces.

Alternatively or additionally, the polymeric coating may couple the proximal stent segment and the distal stent segment to the central stent segment and may limit how far the proximal stent segment and/or the distal stent segment are able to move relative to the central stent segment.

Alternatively or additionally, the polymeric coating may extend from a proximal end of the central stent segment to a distal end of the central stent segment.

Alternatively or additionally, the proximal stent segment may have an inner diameter that is greater than a sum of an outer diameter of the central stent segment and twice a thickness of the polymeric coating, and the distal stent segment may have an inner diameter that is greater than the sum of the outer diameter of the central stent segment and twice the thickness of the polymeric coating.

Alternatively or additionally, the polymeric coating may extend over the proximal stent segment, a central portion of the central stent segment, and the distal stent segment.

Alternatively or additionally, at least a portion of the proximal region of the central stent segment may be uncoated to facilitate drainage.

Alternatively or additionally, the proximal stent segment may have an inner diameter that is greater than an outer diameter of the central stent segment, and the distal stent segment may have an inner diameter that is greater than an outer diameter of the central stent segment.

Alternatively or additionally, the polymeric stent segment may include an annular flange.

Alternatively or additionally, the distal stent segment may include one or more annular flanges.

Alternatively or additionally, the central stent segment may include a braided stent segment, the proximal stent segment may include a braided stent segment, and the distal stent segment may include a braided stent segment.

Another example may be found in a method of forming a medical stent that includes a central stent segment, a proximal stent segment adapted to be positioned over a proximal region of the central stent segment and a distal stent segment adapted to be positioned over the distal region of the central stent segment. The method includes disposing the proximal stent segment, the central stent segment and the distal stent segment on a mandrel with the proximal stent segment spaced a proximal distance from a proximal end of the central stent segment and the distal stent segment is spaced a distal distance from a distal end of the central stent segment. A polymeric coating that extends from the proximal stent segment to the distal stent segment is formed, the polymeric coating spanning between the proximal stent segment and the proximal end of the central stent segment and between the distal end of the central stent segment and the distal stent segment to form a stent assembly. The stent assembly is removed from the mandrel. The proximal stent segment is slide over the proximal region of the central stent segment and the distal stent segment is slide over the distal region of the central stent segment to form the medical stent.

Alternatively or additionally, the proximal distance and the distal distance may each be in a range of 10 to 50 percent of a length of the central stent segment.

Alternatively or additionally, forming the polymeric coating may include forming a silicone coating.

Alternatively or additionally, at least one of the proximal stent segment and the distal stent segment may include an annular flange extending radially outwardly from the proximal stent segment and/or the distal stent segment.

Another example may be found in a medical stent. The medical stent includes a central stent segment including a proximal region, a flared distal region and an intervening center region. A proximal stent segment is adapted to be positioned over the proximal region of the central stent segment. A polymeric coating extends over the proximal stent segment, the flared distal region and the intervening center region of the central stent segment. The polymeric coating is adapted to be doubled over on itself to dispose the proximal stent segment over the proximal region of the central stent segment.

Another example may be found in a medical stent. The medical stent includes a central stent segment including a proximal region and a distal region. A proximal stent segment is adapted to be positioned over the proximal region of the central stent segment. A distal stent segment is adapted to be positioned over the distal region of the central stent segment. A polymeric coating extends over the proximal stent segment, the distal stent segment and at least a portion of the central stent segment, and is adapted to allow the proximal stent segment to float over the proximal region of the central stent segment and to allow the distal stent segment to float over the distal region of the central stent segment. Upon implantation, the proximal stent segment and/or the distal stent segment are adapted to translate relative to the central stent segment in response to peristaltic forces.

Alternatively or additionally, the polymeric coating may couple the proximal stent segment to the central stent segment.

Alternatively or additionally, the polymeric coating may couple the distal stent segment to the central stent segment.

Alternatively or additionally, the polymeric coating may limit how far the proximal stent segment and/or the distal stent segment are able to move relative to the central stent segment.

Alternatively or additionally, the polymeric coating may extend from a proximal end of the central stent segment to a distal end of the central stent segment.

Alternatively or additionally, the proximal stent segment may have an inner diameter that is greater than a sum of an outer diameter of the central stent segment and twice a thickness of the polymeric coating, and the distal stent segment may have an inner diameter that is greater than the sum of the outer diameter of the central stent segment and twice the thickness of the polymeric coating.

Alternatively or additionally, the polymeric coating may extend over the proximal stent segment, a central portion of the central stent segment, and the distal stent segment.

Alternatively or additionally, at least a portion of the proximal region of the central stent segment may be uncoated to facilitate drainage.

Alternatively or additionally, the proximal stent segment may have an inner diameter that is greater than an outer diameter of the central stent segment and the distal stent segment may have an inner diameter that is greater than an outer diameter of the central stent segment.

Alternatively or additionally, the polymeric stent segment may include an annular flange.

Alternatively or additionally, the distal stent segment may include one or more annular flanges.

Alternatively or additionally, the central stent segment may include a braided stent segment.

Alternatively or additionally, the proximal stent segment may include a braided stent segment.

Alternatively or additionally, the distal stent segment may include a braided stent segment.

Another example may be found in a method of forming a medical stent that includes a central stent segment, a proximal stent segment adapted to be positioned over a proximal region of the central stent segment and a distal stent segment adapted to be positioned over the distal region of the central stent segment. The method includes disposing the proximal stent segment, the central stent segment and the distal stent segment on a mandrel with the proximal stent segment spaced a proximal distance from a proximal end of the central stent segment and the distal stent segment is spaced a distal distance from a distal end of the central stent segment. A polymeric coating that extends from the proximal stent segment to the distal stent segment is formed, the polymeric coating spanning between the proximal stent segment and the proximal end of the central stent segment and between the distal end of the central stent segment and the distal stent segment to form a stent assembly. The stent assembly is removed from the mandrel. The proximal stent segment is slide over the proximal region of the central stent segment and the distal stent segment is slid over the distal region of the central stent segment to form the medical stent.

Alternatively or additionally, the proximal distance may be in a range of 10 percent to 50 percent of a length of the central stent segment.

Alternatively or additionally, the distal distance may be in a range of 10 percent to 50 percent of a length of the central stent segment.

Alternatively or additionally, forming the polymeric coating may include forming a silicone coating.

Alternatively or additionally, at least one of the proximal stent segment and the distal stent segment may include an annular flange extending radially outwardly from the proximal stent segment and/or the distal stent segment.

Another example may be found in a medical stent. The medical stent includes a central stent segment including a proximal region, a flared distal region and an intervening center region. A proximal stent segment is adapted to be positioned over the proximal region of the central stent segment. A polymeric coating extends over the proximal stent segment, the flared distal region and the intervening center region of the central stent segment and is adapted to be doubled over on itself to dispose the proximal stent segment over the proximal region of the central stent segment.

The preceding summary is provided to facilitate an understanding of some of the innovative features unique to the present disclosure and is not intended to be a full description. A full appreciation of the disclosure can be gained by taking the entire specification, claims, figures, and abstract as a whole.

### BRIEF DESCRIPTION OF THE DRAWINGS

The disclosure may be more completely understood in consideration of the following description of various examples in connection with the accompanying drawings, in which:

FIG. 1 is a schematic view of an example medical stent;

FIGS. 2A through 2C are schematic views showing an example manufacturing process for making the example medical stent of FIG. 1;

FIGS. 3A through 3D are schematic views of the example medical stent of FIG. 1, shown in an esophageal implantation;

FIG. 4 is a schematic view of an example medical stent;

FIG. 5 is a schematic view of an example medical stent; and

FIG. 6 is a schematic view of an example medical stent.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DESCRIPTION

The following description should be read with reference to the drawings, in which like elements in different drawings are numbered in like fashion. The drawings, which are not necessarily to scale, depict examples that are not intended to limit the scope of the disclosure. Although examples are illustrated for the various elements, those skilled in the art will recognize that many of the examples provided have suitable alternatives that may be utilized.

All numbers are herein assumed to be modified by the term “about”, unless the content clearly dictates otherwise. The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is contemplated that the feature, structure, or characteristic may be applied to other embodiments whether or not explicitly described unless clearly stated to the contrary.

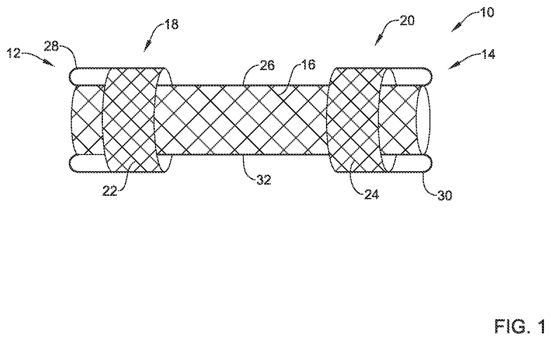

Stents are utilized in a variety of different body lumens, including the vasculature and various parts of the gastrointestinal system, for example. In some instances, particularly in gastrointestinal applications, an implanted stent may be subjected to a variety of different forces. For example, the gastrointestinal system may view the stent as a foreign object that should be expelled. In some instances, some parts of the gastrointestinal system may attempt to force an implanted stent to move in a distal direction. In some instances, some parts of the gastrointestinal system may attempt to force an implanted stent to move in a proximal, or upward and outward, direction. It will be appreciated that some parts of the gastrointestinal system naturally undergo movement such as peristaltic movement in order to cause food and food substances such as partially digested food to move distally through the gastrointestinal system. Stents placed in various parts of the gastrointestinal system, such as but not limited to the esophagus, are subjected to peristaltic movement. FIG. 1 is a schematic view of an example medical stent 10 that is adapted for use in body lumens in which the medical stent 10 will be subjected to various movement forces.

In FIG. 1, the example medical stent 10 extends from a proximal end 12 to a distal end 14, recognizing that these designations are arbitrary, and depend on the orientation in which the medical stent 10 will ultimately be implanted within a body lumen. The medical stent 10 includes a central stent segment 16 having a proximal region 18 and a distal region 20. The medical stent 10 includes a proximal stent segment 22 that is adapted to be positioned over the proximal region 18 of the central stent segment 16. The medical stent 10 includes a distal stent segment 24 that is adapted to be positioned over the distal region 20 of the central stent segment 16. As can be seen, the proximal stent segment 22 and the distal stent segment 24 each have an inner diameter that is greater than an outer diameter of the central stent segment 16. In some instances, the inner diameter of the proximal stent segment 22 and/or the distal stent segment 24 may be spaced radially outward and away from the outer diameter of the central stent segment 16, providing an annular gap therebetween.

The central stent segment 16, the proximal stent segment 22 and the distal stent segment 24 are each distinct elements that are separately formed and then are assembled into the medical stent 10. Thus, the central stent segment 16 may not be directly connected to either the proximal stent segment 22 or the distal stent segment 24. Each of the central stent segment 16, the proximal stent segment 22 and the distal stent segment 24 may each be formed with one or more interwoven filaments. The one or more interwoven filaments of each of the central stent segment 16, the proximal stent segment 22 and the distal stent segment 24 may not be continuous with the one or more interwoven filaments of the other segments. As shown, each of the central stent segment 16, the proximal stent segment 22 and the distal stent segment 24 are each formed as braided stent segments. A variety of braiding patterns may be used in creating the central stent segment 16, the proximal stent segment 22 and the distal stent segment 24. In some instances, one or more of the central stent segment 16, the proximal stent segment 22 and the distal stent segment 24 may be woven stent segments, and in some instances may be a knitted construction. In some instances, one or more of the central stent segment 16, the proximal stent segment 22 and the distal stent segment 24 may be a monolithic tubular construct, such as laser cut stent segments. In some instances, the central stent segment 16 may be dimensioned to fit within a constriction within a body lumen in which the medical stent 10 will be implanted. In some instances, the proximal stent segment 22 and the distal stent segment 24 may each be dimensioned to engage the lumen wall of the body lumen in which the medical stent 10 will be implanted.

The central stent segment 16 may be adapted to move axially (distally and proximally) relative to the proximal stent segment 22 and the distal stent segment 24 in order to accommodate movement such as peristaltic motion within the body lumen without dislodging the medical stent 10. In some instances, the central stent segment 16 may be adapted to move (e.g., move in an axially distal and/or axially proximal direction) in response to applied forces while the proximal stent segment 22 and the distal stent segment 24 remain in place relative to the lumen wall of the body lumen in which the medical stent 10 will be implanted. In some instances, the medical stent 10 includes a polymeric coating 26 that extends over the proximal stent segment 22, the distal stent segment 24 and the central stent segment 16, thereby coupling the proximal stent segment 22 and the distal stent segment 24 with the central stent segment 16. The polymeric coating 26 may extend between the proximal stent segment 22 and the central stent segment 16 thereby coupling the proximal stent segment 22 with the central stent segment 16. The polymeric coating 26 may extend between the distal stent segment 24 and the central stent segment 16 thereby coupling the distal stent segment 24 with the central stent segment 16. In some instances, the polymeric coating 26 may be the only structure connecting the central stent segment 16 with the proximal stent segment 22 and/or distal stent segment 24. In some instances, the polymeric coating 26 may be considered as limiting how far the central stent segment 16 is able to translate relative to the proximal stent segment 22 and the distal stent segment 24.

In some instances, the polymeric coating 26 may be considered as being adapted to allow the proximal stent segment 22 to float over the proximal region 18 of the central stent segment 16 and to allow the distal stent segment 24 to float over the distal region 20 of the central stent segment 16. Upon implantation, the proximal stent segment 22 and/or the distal stent segment 24 may be adapted to translate relative to the central stent segment 16 in response to peristaltic forces while the central stent segment 16 remain in place relative to the lumen wall of the body lumen in which the medical stent 10 is implanted.

In some instances, the polymeric coating 26 includes a proximal inversion region 28 in which the polymeric coating 26 is doubled over on itself in order to allow the proximal stent segment 22 to be positioned over the proximal region 18 of the central stent segment 16. The proximal inversion region 28 may define a proximal most extent of the stent 10, which extends proximal of the central stent segment 16 and the proximal stent segment 22. In some instances, the polymeric coating 26 includes a distal inversion region 30 in which the polymeric coating 26 is doubled over on itself in order to allow the distal stent segment 24 to be positioned over the distal region 20 of the central stent segment 16. The distal inversion region 30 may define a distal most extent of the stent 10, which extends distal of the central stent segment 16 and the distal stent segment 24.

As will be described further herein, the central stent segment 16 may be axially moveable relative to the proximal stent segment 22 and/or the distal stent segment 24 by lengthening the proximal inversion region 28 while shortening the distal inversion region 30 and/or by lengthening the distal inversion region 30 while shortening the proximal inversion region 28. Thus, axial movement of the central stent segment 16 relative to the proximal stent segment 22 and/or the distal stent segment 24 may be accomplished without changing the size (e.g., diameter and/or length) of any of the central stent segment 16, the proximal stent segment 22, and the distal stent segment 24.

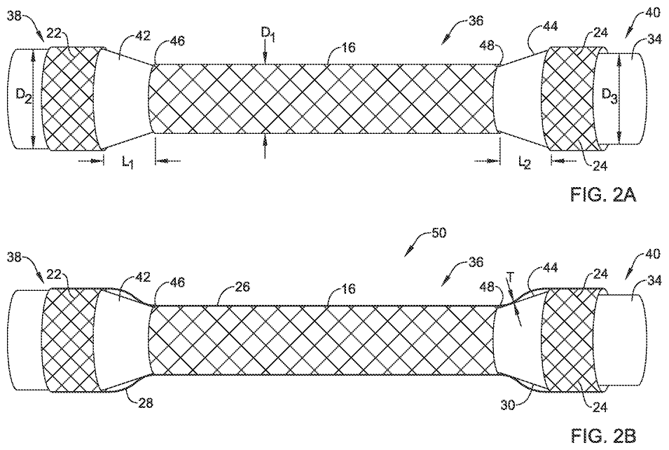

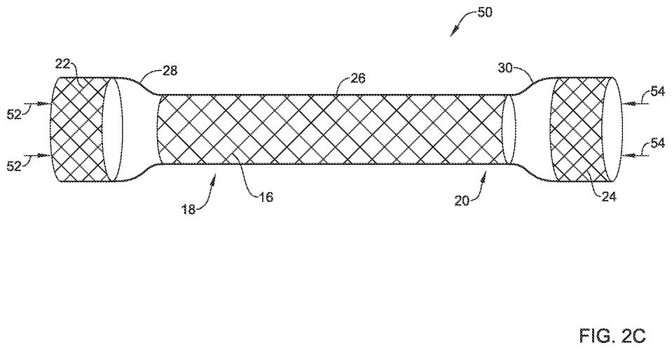

FIGS. 2A through 2C show an example method for forming the medical stent 10. In this example, the central stent segment 16, the proximal stent segment 22 and the distal stent segment 24 have already been manufactured, and are assembled together to form the medical stent 10. The central stent segment 16, the proximal stent segment 22 and the distal stent segment 24 may be braided, woven or laser cut using known techniques. The polymeric coating 26 may be applied using known techniques such as spray coating, dip coating and the like.

In FIG. 2A, the central stent segment 16, the proximal stent segment 22 and the distal stent segment 24 have been positioned on a mandrel 34. It will be appreciated that the mandrel 34 includes a center portion 36 that is dimensioned to accommodate the central stent segment 16, an enlarged diameter first portion 38 that is dimensioned to accommodate the proximal stent segment 22 and an enlarged diameter second portion 40 that is dimensioned to accommodate the distal stent segment 24. The mandrel 34 may include a first tapered portion 42 between enlarged diameter first portion 38 and the center portion 36. The mandrel 34 may include a second tapered portion 44 between the center portion 36 and the enlarged diameter second portion 40.

Several dimensions may be called out with respect to FIG. 2A. The central stent segment 16 has an outer diameter D₁. The proximal stent segment 22 has an inner diameter D₂. The distal stent segment 24 has an inner diameter D₃. The dimensions D₁, D₂ and D₃ will vary, depending on the overall size of the medical stent 10, and the particular intended implantation site for the medical stent 10. In some instances, the inner diameter D₂ of the proximal stent segment 22 and the inner diameter D₃ of the distal stent segment 24 may be selected, relative to the outer diameter D₁ of the central stent segment 16, to allow the proximal stent segment 22 and the distal stent segment 24 to be positioned over the proximal region 18 of the central stent segment 16 and the distal region 20 of the central stent segment 16, respectively, taking into account the thickness of the polymeric coating 26. In some instances, the outer diameter D₁ of the central stent segment 16 may range from 14 millimeters to 25 millimeters for an esophageal, enteral or bariatric application. For more biliary applications, D₁ may range from 5 millimeters to 12 millimeters. In some instances, the inner diameter D₂ of the proximal stent segment 22 may range from 15 millimeters to 35 millimeters. In some instances, such as for a biliary application, D₂ may range from 6 millimeters to 16 millimeters. As an example, the proximal stent segment 22 may be placed outside of a biliary duct and be allowed to get to a larger size than the duct itself. In some instances, the inner diameter D₃ of the stent segment 24 may range from 15 millimeters to 35 millimeters. While the central stent segment 16 is shown as having a constant diameter, in some instances the central stent segment 16 may be tapered.

When placed on the mandrel 34, the proximal stent segment 22 may be spaced apart a distance L₁ from a proximal end 46 of the central stent segment 16. When placed on the mandrel 34, the distal stent segment 24 may be spaced apart a distance L₂ from a distal end 48 of the central stent segment 16. In some instances, L₁ and L₂ may be selected in order to define the relative length of the polymeric coating 26 that spans the distance between the proximal stent segment 22 and the central stent segment 16 and between the central stent segment 16 and the distal stent segment 24, respectively. In some instances, the values of L₁ and L₂ define the lengths of the proximal inversion region 28 and the distal inversion region 30, respectively.

In some instances, L₁ and L₂ can impact how far the central stent segment 16 is allowed to translate relative to the proximal stent segment 22 and the distal stent segment 24 once implanted. In some instances, L₁ and L₂ may be selected to be equal to each other. In some instances, L₁ may be selected to be larger than L₂, if there is a desire to allow relatively greater movement between the central stent segment 16 and the proximal stent segment 22. In some instances, L₂ may be selected to be larger than L₁, if there is a desire to allow relatively greater movement between the central stent segment 16 and the distal stent segment 24. In some instances, L₁ and L₂ may each range from 10 percent to 50 percent of the central stent segment 16. In some instances, controlling the lengths L₁ and L₂ may be considered as controlling or limiting how far the central stent segment 16 is allowed to slide or translate relative to the proximal stent segment 22 and/or the distal stent segment 24, for example.

After placing the central stent segment 16, the proximal stent segment 22 and the distal stent segment 24 in position on the mandrel 34, with appropriate spacing between the proximal stent segment 22 and the central stent segment 16, and between the central stent segment 16 and the distal stent segment 24, the polymeric coating 26 may be applied, as seen in FIG. 2B. The polymeric coating 26 may be applied using known techniques such as spray coating, dip coating, and the like. As noted, the polymeric coating 26 may be applied using any suitable technique such as spray coating or dip coating. The polymeric coating 26 may have a thickness T, as indicated in FIG. 2B. In some instances, the thickness T may be selected in order to provide a clearance between the inner dimensions of the proximal stent segment 22 and the distal stent segment 24 in order to allow the central stent segment 16 to move into and out of the proximal stent segment 22 and the distal stent segment 24. The thickness T of the polymeric coating 26 may be in a range of 10 microns to 500 microns, for example. In some instances, the thickness T of the polymeric coating 26 could be even greater, as long as there is sufficient clearance for the central stent segment 16 to translate relative to the proximal. stent segment 22 and the distal stent segment 24.

In some instances, the thickness T is constant across a length of the medical stent 10. In some instances, the thickness T of the polymeric coating 26 may vary, depending on relative location on the medical stent 10. As an example, the polymeric coating 26 may be thicker within the proximal inversion region 28 and the distal inversion region 30, to better withstand functioning as a living hinge as the central stent segment 16 translates relative to the proximal stent segment 22 and the distal stent segment 24. Alternatively, the polymeric coating 26 may be thinner within the proximal inversion region 28 and the distal inversion region 30, to be more flexible as the central stent segment 16 translates relative to the proximal stent segment 22 and the distal stent segment 24.

As can be seen, the polymeric coating 26 covers all of the central stent segment 16, extending from the proximal end 46 of the central stent segment 16 to the distal end 48 of the central stent segment. In some instances, the inner diameter D₂ of the proximal stent segment 22 is greater than a sum of the outer diameter D₁ of the central stent segment 16 and twice the thickness T of the polymeric coating 26 in order to allow the proximal stent segment 22 to not only fit over the proximal region 18 of the central stent segment 16 but to allow free movement between the central stent segment 16 and the proximal stent segment 22. Similarly, the inner diameter D₃ of the distal stent segment 24 is greater than a sum of the outer diameter D₁ of the central stent segment 16 and twice the thickness T of the polymeric coating 26 in order to allow the distal stent segment 24 to not only fit over the distal region 20 of the central stent segment 16 but to allow free movement between the central stent segment 16 and the distal stent segment 24.

Forming the polymeric coating 26 over the proximal stent segment 22, the central stent segment 16 and the distal stent segment 24 may be considered as forming an assembly 50. Thus, the polymeric coating 26 may connect the proximal stent segment 22, the central stent segment 16 and the distal stent segment 24 together to define the assembly 50 The next step is to remove the assembly 50 from the mandrel 34, as shown in FIG. 2C. In some instances, the assembly 50 may be sufficiently flexible to simply be pulled off the mandrel 34. In some instances, the mandrel 34 may be collapsible in order to allow the assembly 50 to be more easily removed. In some instances, the mandrel 34 may be used in an expanded or inflated configuration as shown in FIGS. 2A and 2B, and removing the assembly 50 from the mandrel 34 simply involves deflating the mandrel 34.

Once the assembly 50 has been removed from the mandrel 34, the proximal stent segment 22 may be moved in a direction indicated by arrows 52 in order to position the proximal stent segment 22 over the proximal region 18 of the central stent segment 16, thereby inverting the proximal inversion region 28 of the polymeric coating 26. The distal stent segment 24 may be moved in a direction indicated by arrows 54 in order to position the distal stent segment 24 over the distal region 20 of the central stent segment 16, thereby inverting the distal inversion region 30 of the polymeric coating 26. The results are the medical stent 10, as shown for example in FIG. 1.

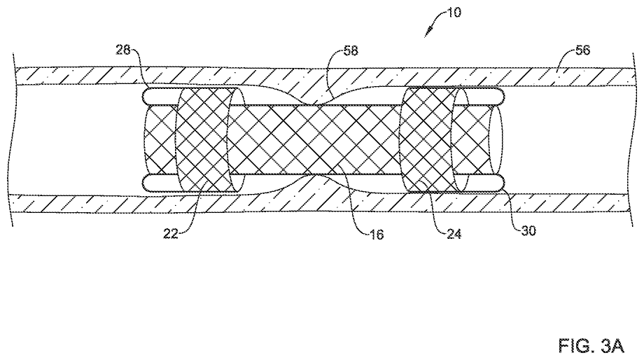

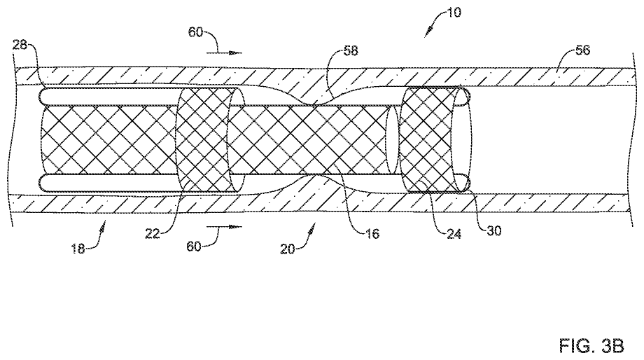

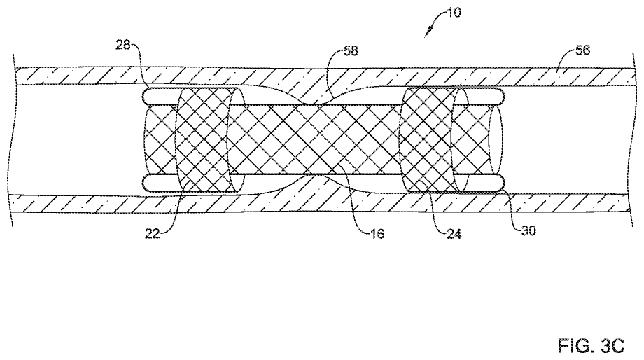

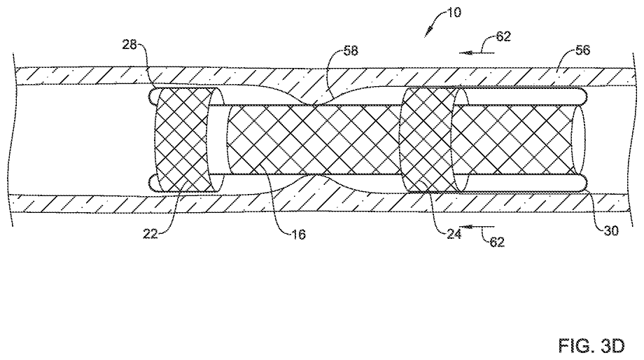

FIGS. 3A through 3D provide a schematic example of how the medical stent 10 resists peristaltic movement when implanted within an esophagus 56 proximate a restriction 58 within the esophagus 56. It will be appreciated that this is merely illustrative, as the medical stent 10 may function similarly within a variety of different body lumens. In FIG. 3A, the medical stent 10 may be considered as being relaxed, with no particular forces causing movement of the central stent segment 16 relative to the proximal stent segment 22 or the distal stent segment 24, and with the proximal inversion region 28 of the polymeric coating 26 equal or substantially equal (within ten percent or so) in length to the distal inversion region 30.

Moving to FIG. 3B, peristaltic forces, indicated by arrows 60, tend to force the proximal stent segment 22 in a distal direction. The polymeric coating 26 decouples the peristaltic forces from the central stent segment 16. As a result of the movement, the proximal inversion region 28 of the polymeric coating 26 has lengthened while the distal inversion region 30 of the polymeric coating 26 has shortened or is no longer inverted. In some instances, the polymeric coating 26 may stretch in response to applied forces, which helps to cause the medical stent to revert to its relaxed configuration when the applied forces are removed, as shown in FIG. 3C.

Moving to FIG. 3D, reverse peristaltic forces (which may be caused by one or more of regurgitation or vomiting) indicated by arrows 62, tend to force the distal stent segment 24 in a proximal direction. The polymeric coating 26 decouples the peristaltic forces from the central stent segment 16. As a result of the movement, the distal inversion region 30 of the polymeric coating 26 has lengthened while the proximal inversion region 28 of the polymeric coating 26 has shortened or is no longer inverted. In some instances, the polymeric coating 26 may stretch in response to applied forces, which helps to cause the medical stent to revert to its relaxed configuration when the applied forces are removed, as shown once again in FIG. 3C.

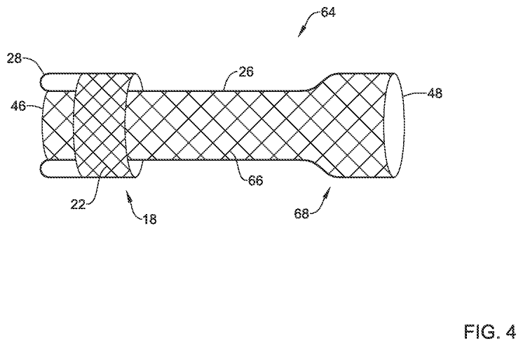

In some instances, it may be sufficient to just have one of the proximal stent segment 22 and the distal stent segment 24 be able to translate relative to the central stent region 16, rather than both. As an example, this may be sufficient in situations where applied anatomical force that may cause migration are unidirectional, or in situations in which a single dynamic element such as the proximal stent segment 22 and proximal inversion region 28 of the polymeric coating 26 are sufficient to resist migration. FIG. 4 is a schematic view of an example medical stent 64 that employs the proximal stent segment 22, joined to a central stent segment 66 via the proximal inversion region 28 of the polymeric coating 26. The central stent segment 66 includes a flared distal region 68 that is dimensioned to engage the lumen wall of the body lumen in which the medical stent 64 will be implanted.

It will be appreciated that manufacture of the medical stent 64 will be similar to that of the medical stent 10, with placing the proximal stent segment 22 on a mandrel spaced an appropriate distance from the central stent segment 66, followed by applying the polymeric coating 26 and subsequently removing from the mandrel and disposing the proximal stent segment 22 over the proximal region 18 of the central stent segment 26, thereby inverting the proximal inversion region 28 of the polymeric coating 26. While shown and described as including the proximal stent segment 22 and the distal flared region 68, it will be appreciated that a medical stent similar to the medical stent 64 may instead include a proximal flared region and the distal stent segment 24.

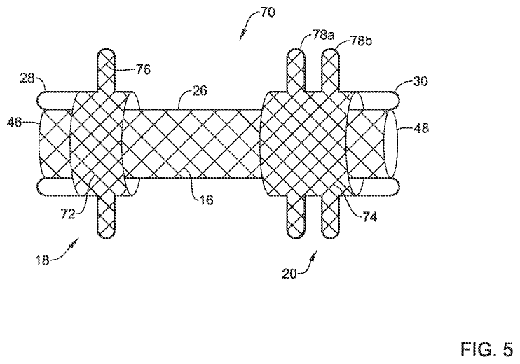

In some instances, the proximal stent segment 22 and/or the distal stent segment 24 may include features intended to improve stability of the medical stent within the anatomy. FIG. 5 is a schematic view of an example medical stent 70. The medical stent 70 includes the central stent segment 16 extending from the proximal end 46 to the distal end 48, and including the proximal region 18 of the central stent segment 16 and the distal region 20 of the central stent segment 16. The medical stent 70 includes a proximal stent segment 72 that is adapted to be disposed over the proximal region 18 of the central stent segment 16 and a distal stent segment 74 that is adapted to be disposed over the distal region 20 of the central stent segment 16.

Manufacture of the medical stent 70 is similar to that of the medical stent 10, with positioning the proximal stent segment 72, the central stent segment 16 and the distal stent segment 74 on a mandrel with appropriate spacing therebetween, followed by forming the polymeric coating 26, removal from the mandrel and then positioning the proximal stent segment 72 over the proximal region 18 of the central stent segment 16, thereby forming the proximal inversion region 28, and positioning the distal stent segment 74 over the distal region 20 of the central stent segment 16, thereby forming the distal inversion region 30.

The proximal stent segment 72 includes an annular flange 76 that extends radially outwardly from the proximal stent segment 72. The distal stent segment 74 includes a first annular flange 78a and a second annular flange 78b, each of which extend radially outwardly from the distal stent segment 74. In some instances, the annular flange 76 may be integrally formed when braiding, weaving or laser cutting the proximal stent segment 72. In some instances, the annular flange 76 may be separately formed and subsequently secured to the proximal stent segment 72. In some instances, the annular flanges 78a and 78b may be integrally formed when braiding, weaving or laser cutting the distal stent segment 74. In some instances, the annular flanges 78a and 78b may be separately formed and subsequently secured to the distal stent segment 74.

While the medical stent 70 is shown having a single annular flange 76 on the proximal stent segment 72, and a pair of annular flanges 78a and 78b on the distal stent segment 74, this is merely illustrative. In some instances, the proximal stent segment 72 and the distal stent segment 74 may each include a single annular flange. In some instances, the proximal stent segment 72 and the distal stent segment 74 may each include a pair of annular flanges. In some instances, having a pair of annular flanges on at least one of the proximal stent segment 72 and the distal stent segment 74 may help with orienting the medical stent 70 within a body lumen, without the medical stent 70 being positioned at an angle within the body lumen.

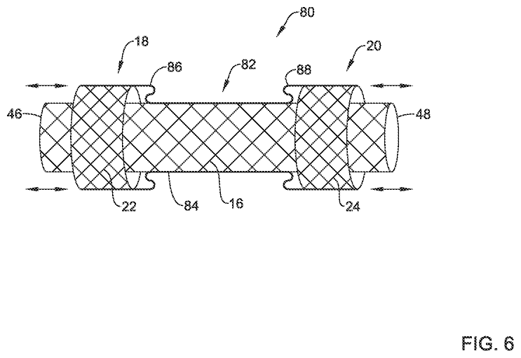

FIG. 6 is a schematic view of an example medical stent 80. The example medical stent 80 is similar to the medical stent 10, but includes a variation on how the polymeric coating is applied. As seen in FIG. 6, the medical stent 80 includes the central stent segment 16, extending from the proximal end 46 to the distal end 48, and including the proximal region 18 of the central stent segment 16 and the distal region 20 of the central stent segment 16. The central stent segment 16 also includes a middle region 82. The proximal stent segment 22 is positioned over the proximal region 18 of the central stent segment 16 and the distal stent segment 24 is positioned over the distal region 20 of the central stent segment 16. The proximal stent segment 22 may be positioned radially outward of and spaced away from the central stent segment 16, providing an annular space therebetween. Similarly, the distal stent segment 24 may be positioned radially outward of and spaced away from the central stent segment 16. The central stent segment 16 may extend into or through the proximal stent segment 22 and/or the central stent segment 16 may extend into or through the distal stent segment 24. In some instances, the central stent segment 16 may extend proximal of the proximal stent segment 22 and/or the central stent segment 16 may extend distal of the distal stent segment 24.

The medical stent 80 includes a polymeric coating 84 that extends over the proximal stent segment 22, the middle region 82 of the central stent segment 16 and the distal stent segment 24. The polymeric coating 84 does not extend to the proximal end 46 or the distal end 48 of the central stent segment 16, and thus does not include inversion regions at the further extents of the stent 80. Rather, the polymeric coating 84 includes a proximal hinge region 86 and a distal hinge region 88 that allow for axial movement of the proximal stent segment 22 and the distal stent segment 24 relative to the central stent segment 16 in response to peristaltic forces. In some instances, one or more ends of the central stent segment 16 may be uncovered, in order to facilitate drainage.

The proximal hinge region 86 of the polymeric coating 84 may extend between the proximal stent segment 22 and the central stent segment 16 thereby coupling the proximal stent segment 22 with the central stent segment 16. The distal hinge region 88 of the polymeric coating 84 may extend between the distal stent segment 24 and the central stent segment 16 thereby coupling the distal stent segment 24 with the central stent segment 16. In some instances, the polymeric coating 84 may be the only structure connecting the central stent segment 16 with the proximal stent segment 22 and/or distal stent segment 24. In some instances, the polymeric coating 84 may be considered as limiting how far the central stent segment 16 is able to translate relative to the proximal stent segment 22 and the distal stent segment 24.

In some instances, the inner diameter D₂ (see FIG. 2A) of the proximal stent segment 22 may be greater than the outer diameter D₁ of the central stent segment 16. In some instances, the inner diameter D₃ of the distal stent segment 24 may be greater than the outer diameter D₁ of the central stent segment 16. It will be appreciated that with the configuration of the polymeric coating 84 shown in FIG. 6, the inner diameter D₂ of the polymeric stent segment 22 and the inner diameter D₃ of the distal stent segment 24 may be less than the corresponding diameters shown for the medical stent 10, since the medical stent 80 does not have to account for the thickness of the polymeric coating 84 because the polymeric coating 84 does not extend between the outer surface of the central stent segment 16 and the inner surfaces of the proximal stent segment 22 and the distal stent segment 24. The medical stent 80 is adapted to isolate the central stent segment 16 from applied forces.

The materials that can be used for the various components of the medical stent(s), the mandrel, and the various elements thereof disclosed herein may include those commonly associated with medical devices and mandrels. For simplicity purposes, the following discussion refers to the apparatus. However, this is not intended to limit the devices and methods described herein, as the discussion may be applied to other elements, members, components, or devices disclosed herein, such as, but not limited to, the medical stent, the mandrel, the filaments, the anti-migration loops, the covering, and/or elements or components thereof.

In some embodiments, the apparatus, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-NR and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the apparatus, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the apparatus in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the apparatus to achieve the same result.

In some embodiments, a degree of Magnetic Resonance Imaging (MRI) compatibility is imparted into the apparatus and/or other elements disclosed herein. For example, the apparatus, and/or components or portions thereof, may be made of a material that does not substantially distort the image and create substantial artifacts (e.g., gaps in the image). Certain ferromagnetic materials, for example, may not be suitable because they may create artifacts in an MRI image. The apparatus, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-NR and the like), nitinol, and the like, and others.

In some embodiments, the apparatus and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); antiproliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antincoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, antiplatelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

Having thus described several illustrative embodiments of the present disclosure, those of skill in the art will readily appreciate that yet other embodiments may be made and used within the scope of the claims hereto attached. It will be understood, however, that this disclosure is, in many respects, only illustrative. Changes may be made in details, particularly in matters of shape, size, arrangement of parts, and exclusion and order of steps, without exceeding the scope of the disclosure. The disclosure's scope is, of course, defined in the language in which the appended claims are expressed.

What is claimed is:

1. A medical stent, comprising:
a central stent segment including a proximal region and a distal region;
a proximal stent segment adapted to be positioned over the proximal region of the central stent segment;
a distal stent segment adapted to be positioned over the distal region of the central stent segment; and
a polymeric coating extending over the proximal stent segment, the distal stent segment and at least a portion of the central stent segment, the polymeric coating adapted to allow the proximal stent segment to float over the proximal region of the central stent segment and to allow the distal stent segment to float over the distal region of the central stent segment;
wherein upon implantation, the proximal stent segment and/or the distal stent segment are adapted to translate relative to the central stent segment in response to peristaltic forces.

2. The medical stent of claim 1, wherein the polymeric coating couples the proximal stent segment to the central stent segment.

3. The medical stent of claim 1, wherein the polymeric coating couples the distal stent segment to the central stent segment.

4. The medical stent of claim 1, wherein the polymeric coating limits how far the proximal stent segment and/or the distal stent segment are able to move relative to the central stent segment.

5. The medical stent of claim 1, wherein the polymeric coating extends from a proximal end of the central stent segment to a distal end of the central stent segment.

6. The medical stent of claim 5, wherein:
the proximal stent segment has an inner diameter that is greater than a sum of an outer diameter of the central stent segment and twice a thickness of the polymeric coating; and
the distal stent segment has an inner diameter that is greater than the sum of the outer diameter of the central stent segment and twice the thickness of the polymeric coating.

7. The medical stent of claim 2, wherein the polymeric coating extends over the proximal stent segment, a central portion of the central stent segment, and the distal stent segment.

8. The medical stent of claim 7, wherein at least a portion of the proximal region of the central stent segment is uncoated to facilitate drainage.

9. The medical stent of claim 7, wherein:
the proximal stent segment has an inner diameter that is greater than an outer diameter of the central stent segment; and
the distal stent segment has an inner diameter that is greater than an outer diameter of the central stent segment.

10. The medical stent of claim 1, wherein the polymeric stent segment includes an annular flange.

11. The medical stent of claim 1, wherein the distal stent segment includes one or more annular flanges.

12. The medical stent of claim 1, wherein the central stent segment comprises a braided stent segment.

13. The medical stent of claim 1, wherein the proximal stent segment comprises a braided stent segment.

14. The medical stent of claim 1, wherein the distal stent segment comprises a braided stent segment.

15. A method of forming a medical stent that includes a central stent segment, a proximal stent segment adapted to be positioned over a proximal region of the central stent segment and a distal stent segment adapted to be positioned over the distal region of the central stent segment, the method comprising:
disposing the proximal stent segment, the central stent segment and the distal stent segment on a mandrel with the proximal stent segment spaced a proximal distance from a proximal end of the central stent segment and the distal stent segment is spaced a distal distance from a distal end of the central stent segment;
forming a polymeric coating that extends from the proximal stent segment to the distal stent segment, the polymeric coating spanning between the proximal stent segment and the proximal end of the central stent segment and between the distal end of the central stent segment and the distal stent segment to form a stent assembly;
removing the stent assembly from the mandrel;
sliding the proximal stent segment over the proximal region of the central stent segment and sliding the distal stent segment over the distal region of the central stent segment to form the medical stent.

16. The method of claim 15, wherein the proximal distance is in a range of 10 percent to 50 percent of a length of the central stent segment.

17. The method of claim 15, wherein the distal distance is in a range of 10 percent to 50 percent of a length of the central stent segment.

18. The method of claim 15, wherein forming the polymeric coating comprises forming a silicone coating.

19. The method of claim 15, wherein at least one of the proximal stent segment and the distal stent segment includes an annular flange extending radially outwardly from the proximal stent segment and/or the distal stent segment.

20. A medical stent, comprising:
a central stent segment including a proximal region, a flared distal region and an intervening center region;
a proximal stent segment adapted to be positioned over the proximal region of the central stent segment; and
a polymeric coating extending over the proximal stent segment, the flared distal region and the intervening center region of the central stent segment;
wherein the polymeric coating is adapted to be doubled over on itself to dispose the proximal stent segment over the proximal region of the central stent segment.
